(19) United States
(12) Patent Application Publication
(10) Pub. No.: US 2014/0273123 A1
(43) Pub. Date: Sep. 18, 2014

(54) Method for production of n-propanol and other C3-carbon containing products from syngas by symbiotic arrangement of C1-fixing and C3-producing anaerobic microorganism cultures

(71) Applicants: Richard Tobey, St. Charles, IL (US); Rathin Datta, Chicago, IL (US); Michael Enzien, Lisle, IL (US); Robert Hickey, Okemos, MI (US); William Levinson, Naperville, IL (US)

(72) Inventors: Richard Tobey, St. Charles, IL (US); Rathin Datta, Chicago, IL (US); Michael Enzien, Lisle, IL (US); Robert Hickey, Okemos, MI (US); William Levinson, Naperville, IL (US)

(21) Appl. No.: 13/802,916

(22) Filed: Mar. 14, 2013

(51) Int. Cl. C12P 7/04

(52) U.S. Cl. CPC C12P 7/04
USPC 435/157; 435/294.1

(57) ABSTRACT

This invention provides methods and systems for the production of propanol. Specifically, the methods and systems of the present invention use symbiotic arrangement of anaerobic microorganism cultures for the production of propanol from syngas.

### FIELD OF THE INVENTION

The invention provides methods and systems for production of n-propanol and other C3-containing products from syngas using a symbiotic arrangement of C1-fixing and C3-producing anaerobic microorganism cultures.

### BACKGROUND OF THE INVENTION

Propanol is a solvent used industrially, but more importantly, it can be readily dehydrated to produce propylene which is the second largest chemical commodity in the world with production of >70 million tons/per year. Currently propylene is produced mainly by steam-cracking of naphtha or liquid petroleum gas or fluid catalytic cracking of gasoils in very large installations as a secondary product. The steam-cracking is a process that makes majorly ethylene and many other co-products, such as butylenes, butadiene and pyrolysis gasoline all of which need to be purified and to be utilized simultaneously. Other ways to make propylene is in a refinery FCC (fluid catalytic cracking) where propylene is a byproduct from heavy gasoil cracking in proportions between 3 and 15 wt %. Propylene can also be produced by catalytic dehydrogenation of propane. Still another way to make propylene is via metathesis of butenes with ethylene.

Since many centuries, simple sugars are being fermented into ethanol with the help of saccharomyces cerevisae. The last decade's new routes starting from cellulose and hemicelluloses have been developed to ferment more complex carbohydrates into ethanol. Hereto, the carbohydrates need to be unlocked from the lignocellulosic biomass. Biomass consists approximately of 30% cellulose, 35% hemicelluloses and 25% lignin. The lignin fraction cannot be valorised as ethanol, because of its aromatic nature but can only be used as energy source which present in many cases an excess for running an industrial plant.

Several microorganisms are able to use one-carbon compounds as carbon source and some even as energy source. Carbon dioxide is an important carbon source for phototrophs, sulfate reducers, methanogens, acetogens and chemolithotrophic microorganisms. There are essentially four systems to fix CO₂: (1) the Calvin cycle [CO₂ fixing enzyme: ribulose-1,5-bisphosphate carboxylase], (2) the reductive citric acid cycle [CO₂ fixing enzymes: 2-oxoglutarate synthase, isocitrate dehydrogenase, pyruvate synthase], (3) the acetyl-CoA pathway [CO₂ fixing enzyme: acetyl-CoA synthase, linked to CO-dehydrogenase] and (4) the 3-hydroxypropionate cycle [CO₂ fixing enzyme: acetyl-CoA carboxylase, propionyl-CoA carboxylase] (“Structural and functional relationships in Prokaryotes”, L. Barton, Springer 2005; “Carbon monoxide-dependent energy metabolism in anaerobic bacteria and archaea”, E. Oelgeschelager, M. Rother, Arch. Microbiol., 190, p. 257, 2008; “Life with carbon monoxide”, S. Ragsdale, Critical Reviews in Biochem. and Mol. Biology, 39, p. 165, 2004). Several microorganisms can also use carbon monoxide:

Bacteria:
Acetogens (like Acetobacterium woodii, Clostridium pasteurianum etc)
Carboxydotrophs (like Alcaligenes carboxydus, Bacillus schlegelii, Pseudomonas carboxydoflava, Pseudomonas compransori)
Methanotrophs (like Pseudomonas methanica, Methylosinus methanica, Methylococcus capsulatus)
Nitrogen fixers (like Azomonas B1, Azospirillum lipoferum, Bradyrhizobium japonicum)
Phototrophs (like Rhodocyclus gelatinosa, Rhodospirillum rubrum, Spirulina platensis)
Sulfate reducers (like Desulfobacterium autotrophicum, Desulfotomaculum acetoxidans, Desulfovibrio desulfuricans, Desulfovibrio vulgaris)

Archaea:
Methanogens (like Methanobacterium, thermoautotrophicum, Methanosarcina barkeri, Methanothrix soehngenii)

Carboxydotrophs oxidize CO into CO₂ using a molybdenum-containing CO-dehydrogenase and use further the Calvin cycle to fix CO₂. Acetogens can interconvert CO—CO₂ using a Nickel-iron-containing CO-dehydrogenase. This CO-dehydrogenase is linked to an Acetyl-CoA synthase that fixes CO₂ in the Wood-Ljungdahl pathway.

Recently more efficient routes that produce synthesis gas from carbon-containing materials and that subsequently is fermented into ethanol are being developed (“Bioconversion of synthesis gas into liquid or gaseous fuels”, K. Klasson, M. Ackerson, E. Clausen, J. Gaddy, Enzyme and Microbial Technology, 14(8), p. 602, 1992; “Fermentation of Biomass-Generated Producer Gas to Ethanol”, R. Datar, R. Shenkman, B. Cateni, R. Huhnke, R. Lewis, Biotechnology and Bioengineering, 86 (5), p. 587, 2004; “Microbiology of synthesis gas fermentation for biofuel production”, A. Hemstra, J. Sipma, A. Rinzema, A. Stams, Current Opinion in Biotechnology, 18, p. 200, 2007; “Old Acetogens, New Light”, H. Drake, A. Göβner, S. Daniel, Ann. N.Y. Acad. Sci. 1125: 100-128, 2008). Synthesis gas can be produced by gasification of the whole biomass without need to unlock certain fractions. Synthesis gas can also be produced from other feedstock via gasification: (i) coal, (ii) municipal waste (iii) plastic waste, (iv) petcoke and (v) liquid residu's from refineries or from the paper industry (black liquor). Synthesis gas can also be produced from natural gas via steamreforming or autothermal reforming (partial oxidation). For conventional methanol synthesis, higher alcohol synthesis or Fischer-Tropsch a ratio of hydrogen to carbon monoxide of about 2 is required. In case of gasification of hydrogen-poor feedstock this ratio will be below 1 and hence a watergas shift (CO+H₂O→CO₂+H₂) is required to adjust the ratio. The biochemical pathway to transform synthesis gas into ethanol is much less stringent regarding the hydrogen to carbon monoxide ratio.

The biochemical pathway of synthesis gas conversion is described by the Wood-Ljundahl Pathway. Fermentation of syngas offers several advantages such as high specificity of biocatalysts, lower energy costs (because of low pressure and low temperature bioconversion conditions), greater resistance to biocatalyst poisoning and nearly no constraint for a preset H₂ to CO ratio (“Reactor design issues for synthesis-gas fermentations” M. Bredwell, P. Srivastava, R. Worden, Biotechnology Progress 15, 834-844, 1999; “Biological conversion of synthesis gas into fuels”, K. Klasson, C. Ackerson, E. Clausen, J. Gaddy, International Journal of Hydrogen Energy 17, p. 281, 1992). Acetogens are a group of anaerobic bacteria able to convert syngas components, like CO, CO₂ and H₂ to acetate via the reductive acetyl-CoA or the Wood-Ljungdahl pathway.

Several anaerobic bacteria have been isolated that have the ability to ferment syngas to ethanol, acetic acid and other useful end products. Clostridium ljungdahlii and Clostridium autoethanogenum, were two of the first known organisms to convert CO, CO₂ and H₂ to ethanol and acetic acid. Commonly known as acetogens, these microorganisms have the ability to reduce CO₂ to acetate in order to produce required energy and to produce cell mass. The overall stoichiometry for the synthesis of ethanol using three different combinations of syngas components is as follows (J. Vega, S. Prieto, B. Elmore, E. Clausen, J. Gaddy, “The Biological Production of Ethanol from Synthesis Gas”, Applied Biochemistry and Biotechnology, 20-1, p. 781, 1989):

6CO+3H₂O→CH₃CH₂OH+4CO₂

2CO₂+6H₂→CH₃CH₂OH+3H₂O

6CO+6H₂→2CH₃CH₂OH+2CO₂

Acetogenic bacteria are obligate anaerobes that utilize the acetyl-CoA pathway as their predominant mechanism for the reductive synthesis of acetyl-CoA from CO₂ (Drake, H. L. (1994). Acetogenesis. New York: Chapman & Hall). This group of microorganisms is even more versatile in the sense that they can use simple gases like CO₂/H₂ and CO as well as sugars, carboxylic acids, alcohols and amino acids.

Clostridium ljungdahlii, one of the first autotrophic microorganism known to ferment synthesis gas to ethanol was isolated in 1987, as an acetogen favours the production of acetate during its active growth phase (acetogenesis) while ethanol is produced primarily as a non-growth-related product (solventogenesis) (“Biological conversion of synthesis gas into fuels”, K. Klasson, C. Ackerson, E. Clausen, J. Gaddy, International Journal of Hydrogen Energy 17, p. 281, 1992).

Eubacterium limosum is an acetogen, isolated from habitats like the human intestine, rumen, sewage and soil, exhibits high growth rate under high CO concentrations producing acetate, ethanol, butyrate and isobutyrate (I. Chang, B. Kim, R. Lovitt, J. Bang, “Effect of CO partial pressure on cell-recycled continuous CO fermentation by Eubacterium limosum KIST612”, Process Biochemistry, 37(4), p. 411, 2001).

Peptostreptococcus productus is a mesophilic, gram-positive anaerobic coccus, found in the human bowel and is capable of metabolizing CO₂/H₂ or CO to produce acetate (W. Lorowitz, M. Bryant, “Peptostreptococcus productus Strain That Grows Rapidly with CO as the Energy-Source”, Applied and Environmental Microbiology, 47(5), p. 961, 1984).

Clostridium autoethanogenum is a strictly anaerobic, gram-positive, spore-forming, rod-like, motile bacterium which metabolizes CO to form ethanol, acetate and CO₂ as end products, beside it ability to use CO₂ and H₂, pyruvate, xylose, arabinose, fructose, rhamnose and L-glutamate as substrates (J. Abrini, H. Naveau, E. Nyns, “Clostridium autoethanogenum, Sp-Nov, an Anaerobic Bacterium That Produces Ethanol from Carbon-Monoxide”, Archives of Microbiology, 161(4), p. 345, 1994).

Clostridium carboxidivorans P7 is a solvent-producing anaerobe, which was isolated from the sediment of an agricultural settling lagoon. It is motile, gram-positive, spore-forming and primarily acetogenic, forming acetate, ethanol, butyrate, and butanol as end-products. (J. Liou, D. Balkwill, G. Drake, R. Tanner, “Clostridium carboxidivorans sp. nov., a solvent-producing clostridium isolated from an agricultural settling lagoon, and reclassification of the acetogen Clostridium scatologenes strain SL1 as Clostridium drakei sp. nov.”, International Journal of Systematic and Evolutionary Microbiology, 55(5), p. 2085, 2005).

Acetogens are obligate anaerobic bacteria that use the reductive acetyl-CoA pathway as their predominant (i) mechanism for the reductive synthesis of acetyl-CoA from CO₂, (ii) terminal electron-accepting, energy-conserving process, and (iii) mechanism for the synthesis of cell carbon from CO₂″ (Drake, H. L. (1994). Acetogenesis. New York: Chapman & Hall). Like other anaerobes, acetogens require a terminal electron acceptor different from oxygen. In the acetyl-CoA pathway, CO₂ serves as an electron acceptor and H₂ serves as the electron donor. The synthesis of acetyl-CoA from CO₂ and H₂ requires an 8-electron reduction of CO₂ involving the following three steps:

Formation of the carbonyl precursor of acetyl-CoA
Formation of the methyl precursor of acetyl-CoA
Condensation of the above two precursors to form acetyl-CoA.

Anaerobic acetogenic microorganisms offer a viable route to convert waste gases, such as syngas, to useful products, such as ethanol, via a fermentation process. Such bacteria catalyze the conversion of H₂ and CO₂ and/or CO to acids and/or alcohols with higher specificity, higher yields and lower energy costs than can be attained by traditional production processes. While many of the anaerobic microorganisms utilized in the fermentation of ethanol also produce a small amount of propanol as a by-product, to date, no single anaerobic microorganism has been described that can utilize the fermentation process to produce high yields of propanol.

Therefore a need in the art remains for methods using microorganisms in the production of propanol using indirect fermentation.

### SUMMARY OF THE INVENTION

A method has been discovered for producing propanol and/or propionic acid by exposing gaseous substrates of carbon monoxide and/or carbon dioxide and hydrogen to a C1-fixing microorganism, in a first fermentation zone, under conditions effective to convert the gaseous substrate into ethanol or acetate; and passing ethanol and/or acetate that was produced in the first fermentation zone to a C3-producing microorganism contained in a second fermentation zone under conditions effective to convert the ethanol and/or acetate to propionate. In most cases the C3-producing microorganism is a propionogen. Also in most cases the second fermentation zone produces propionate that passes to the first fermentation zone to produce propanol. In addition the gaseous is typically syngas.

In a modified form the method the C1-fixing microorganism are maintained under planktonic conditions and the C3-fixing microorganism is maintained as cells fixed on a support. The fixed support may take the form of a membrane defining pores that retain the cell therein.

In an alternate form there is disclosed an anaerobic symbiotic system for conversion of syngas to propanol or/and to propionic acid, the system comprising syngas, culture media, a C1-fixing microorganism in a first fermentation zone, a C3-producing microorganism in a second fermentation zone, a CO₂ and H₂ source and a transfer conduit for exchanging culture media between the C1-fixing microorganism in the first fermentation zone and the C3-producing microorganism in the second fermentation zone. Again in most cases the C3-producing microorganism is a propionogen.

### BRIEF DESCRIPTION OF THE DRAWINGS

These and other objects, features, and embodiments of the invention will be better understood from the following detailed description taken in conjunction with the drawings, wherein:

FIG. 1 is a schematic diagram of an embodiment of the symbiotic association of anaerobic microorganism cultures of the invention. The C1-fixing microorganism produces ethanol and acetate from syngas. The symbiotic C3-producing microorganism coverts the ethanol, acetate and (secondarily H₂/CO/CO₂) to C3-containing products, namely propionate and propanol. The C1-fixing microorganism also converts the propionate to propanol, which becomes the primary end product.

FIG. 2 is a detailed illustration of the methylmalonyl-succinate pathway used by anaerobic microorganisms for C3 (propionate) production.

FIG. 3 is a detailed illustration of the lactate-acrylate pathway used by anaerobic microorganisms for C3 (propionate/propanol) production.

FIG. 4 shows one embodiment of an arrangement of the fermentation zones of the present invention, where the C1-fixing anaerobic microorganism is fermented in a planktonic fermentation reactor and the C3 producing anaerobic microorganism is fermented in a membrane fermentation reactor.

### DETAILED DESCRIPTION OF THE INVENTION

The invention provides methods for the production of propanol and other C3-containing products from syngas by a symbiotic arrangement of anaerobic microorganism cultures. In other aspects, the invention provides anaerobic systems for conversion of syngas to propanol.

As used herein, synthesis gas (syngas) is a gas containing carbon monoxide, carbon dioxide and frequently hydrogen. “Syngas” includes streams that contain carbon dioxide in combination with hydrogen and that may include little or no carbon monoxide. “Syngas” may also include carbon monoxide gas streams that may have little or no hydrogen.

As used herein, the term “symbiotic” refers to the association of two or more different types (e.g. organisms, populations, strains, species, genera, families, etc.) of anaerobic microorganisms which are capable of forming a tightly associated metabolic symbiosis.

In an embodiment of the invention illustrated in FIG. 1, two types of anaerobic microorganism are utilized to create the symbiotic association for production of propanol. The first type of microorganism in the symbiotic association is a primary C1-fixing microorganism, which utilizes syngas as the sole carbon and electron source and produces ethanol and acetate as the dissimilatory metabolite products. The second type of microorganism in the symbiotic association is capable of growing on the dissimilatory metabolites of the C1-fixing microorganism (ethanol and acetate) as its sole carbon and/or electron source to produce a C3-carbon molecule, such as propanol or propionic acid, as its primary product or together with syngas (as additional carbon and/or electron source) convert the metabolites of the C1-carbon fixing microorganism to C3-carbon molecules. This second microorganism shall be referred to herein as the C3-producing microorganism. Advantageously, the C1-fixing microorganism may also be capable of converting the propionate produced by the C3-producing microorganism into propanol.

The C1-fixing microorganisms of the invention are also homoacetogens. Homoacetogens have the ability, under anaerobic conditions, to produce acetic acid and ethanol from the substrates, CO+H₂O, or H₂+CO₂ or CO+H₂+CO₂. The CO or CO₂ provide the carbon source and the H₂ or CO provide the electron source for the reactions producing acetic acid and ethanol. The primary product produced by the fermentation of CO and/or H₂ and CO₂ by homoacetogens is ethanol according to the following reactions so that the C1 fixing microorganisms are acting as solventogenic homoacetogens using the acetyl-CoA pathway:

6CO+3H₂O→C₂H₅OH+4CO₂

6H₂+2CO₂→C₂H₅OH+3H₂O

Homoacetogens may also produce acetate. Acetate production occurs via the following reactions:

4CO+2H₂O→CH₃COOH+2CO₂

4H₂+2CO₂→CH₃COOH+2H₂O

C1-fixing microorganisms suitable for use in the inventive method include, without limitation, homoacetogens such as Clostridium ljungdahlii, Clostridium autoethanogenum, Clostridium ragsdalei, and Clostridium coskatii. Additional C1 fixing microorganisms that are suitable for the invention include Alkalibaculum bacchi, Clostridium thermoaceticum, and Clostridium aceticum.

Pathways for the production of oxygenates having three carbons: Propionic acid production: Propionibacterium species (Propionibacterium acidipropionici, Propionibacterium acnes, Propionibacterium cyclohexanicum, Propionibacterium freudenreichii, Propionibacterium freudenreichii shermanii) and several other anaerobic bacteria such as Desulfobulbus propionicus, Pectinatus frisingensis, Pelobacter propionicus, Veillonella, Selenomonas, Fusobacterium and Clostridium, in particular Clostridium propionicum, produce propionic acid as a main fermentation product (Playne M., “Propionic and butyric acids”, In: Moo-Young M, editor. Comprehensive biotechnology, New York: Pergamon Press, vol 3, p 731-759, 1985; Seshadri N, Mukhopadhyay S., “Influence of environmental parameters on propionic acid upstream bioprocessing by Propionibacterium acidi-propionici”, J. Biotechnology 29, p. 321-328, 1993). In swiss-type cheeses, propionibacteria consume lactate and produce propionic acid, acetic acid, and CO2. In general, a broad range of substrates can be converted into propionic acid, like glucose, lactose, sucrose, xylose, glycerol and lactate. Propionibacteria are Gram-positive, non-motile, non-sporulating, short-rodshaped, mesophilic anaerobes. The genus of Propionibacterium, belonging to the class of high G+C actinobacteria is divided into two groups: the “cutaneous” and the “dairy” Propionibacteria, based on their habitat (Stackebrandt, E., Cummins, C., Johnson, J., “The Genus Propionibacterium”, in The Prokaryotes, E. Balows, H. Truper, M. Dworkin, W. Harder, K. Scheifer, eds., 2006).

Dicarboxylic Pathway:

Propionibacteria convert carbon sources to produce propionic acid as a main product via the mainly dicarboxylic acid pathway (also called the Wood-Werkman cycle or the methyl-malonyl-CoA pathway), as shown in FIG. 2. Glycolysis pathway catabolyses glucose into phosphoenolpyruvate (PEP), an energy-rich metabolite. Two alternative glycolysis pathways exist: Embden-Meyerhorf-Parnaz (EMP) pathway and Hexose Monophosphate (HMP) pathway. In the EMP pathway, 1 mole of glucose is converted into 2 moles of PEP and 2 moles of NADH, while in the HMP pathway 1 mole of glucose provides 5/3 moles of PEP and 11/3 moles of NADH. PEP is further converted into two possible intermediates, pyruvate and oxaloacetate. The majority of PEP is converted into pyruvate whereas the remaining PEP is converted into oxaloacetate. For pyruvate production, 1 mole of PEP is converted into 1 mole of pyruvate and 1 mole of ATP obtained from a transfer of one phosphoryl moiety from PEP to ADP. The total ATP obtained from the EMP and HMP pathways per mole of glucose is 2 and 5/3 moles, respectively. Glycolysis via the EMP pathway provides a lower amount of NADH (EMP: HMP=2:11/3) but a higher amount of ATP (EMP:HMP=2:5/3). The ratio of EMP to HMP pathway contribution in glycolysis is dependent on propionibacterium species, substrates and fermentation conditions. At the pyruvate node, pyruvate is directed toward three main pathways. Most of pyruvate is converted into propionic acid via the Wood-Werkman cycle. Some of pyruvate converts into acetate while some is incorporated into biomass. In the propionate formation pathway, pyruvate enters the Wood-Werkman cycle, via a transcarboxylation of a carboxyl-moiety from methylmalonyl-CoA to pyruvate, catalysed by oxaloacetate transcarboxylase in a coupled reaction of pyruvate to oxaloacetate and methylmalonyl CoA to propionyl CoA. In this coupled reaction, the carboxyl group transferred from methylmalonyl CoA to pyruvate to form propionyl CoA and oxaloacetate is never released from the reaction or no exchange between this carboxyl group with the dissolved CO2 in the fermentation broth is observed (Wood H G., “Metabolic cycles in the fermentation of propionic acid”, in Current Topics in Cellular regulation, Estabrook and Srera R W, eds., New York: Academic Press. vol 18, p 225-287, 1981). Because of this transcarboxylation reaction, CO₂ fixation is minimal and only used to produce catalytic amounts of oxaloacetate to reinitiate the cycle when for instance succinate accumulates as end-product. Under such circumstances, oxaloacetate is generated by condensation of CO₂ with phosphoenolpyruvate catalysed by a PEP carboxylase. Susequently, oxaloacetate is converted into malate by malate dehydrogenase, malate into fumarate by fumarase and further fumarate to succinate, catalyzed by succinate dehydrogenase. After that succinate is converted into succinyl-CoA, which is then converted into methylmalonyl-CoA. Methylmalonyl-CoA is converted into propionyl-CoA by oxaloacetate transcarboxylase. At the end of the cycle, propionyl-CoA is converted into propionate along with a coupled reaction of succinate to succinyl-CoA, catalysed by propionyl-CoA: succinate transferase. After 1 mole of pyruvate enters the Wood-Werkman cycle, 1 mole of propionate, 2 moles of NAD+, and 1 mole of ATP are generated. Beside propionic acid as main fermentation product, produced in the Wood-Werkman cycle, also NAD+ regeneration for glycolysis occurs in this cycle.

In acetate branch pathway, pyruvate converts to acetyl-CoA and CO₂, catalyzed by pyruvate dehydrogenase complex. Acetyl-CoA is converted into acetyl-phosphate by phosphotransacetylase and further acetyl-phosphate to acetate, catalyzed by acetate kinase. In the acetate branch pathway, 1 mole of acetate, CO₂, NADH, and ATP are obtained from 1 mole of pyruvate. Propionic acid production is usually accompanied by the acetate formation as a major ATP production route supplying energy for cellular metabolism. The following equations represent a theoretical formulation of propionic acid fermentation from glucose or lactate (P. Piveteau, Lait, 79, p. 23, 1999):

1.5 glucose+6Pi+6ADP→2 propionate+acetate+CO₂+2H₂O+6ATP

3 lactic acid+3Pi+3ADP→2 propionate+acetate+CO₂+2H₂O+3ATP

According to these equations, the theoretical maximum yield from glucose is 66.7 C-mole % or 54.8 wt % of propionic acid, 22.2 C-mole % or 22 wt % of acetic acid, 11.1 C-mole % or 17 wt % of CO₂. The theoretically propionic acid to acetic acid (P/A) molar ratio is 2:1. A shift in the metabolic pathway towards the production of propionic acid can be accomplished by using carbon sources with higher reductive level (shift from heterofermentative to homofermentative acid production). A higher reductive level of substrate can cause significant increase in the P/A ratio due to the intracellular NADH/NAD+ balance. A better efficiency of propionic acid production from glycerol could be expected because of its higher reduction level compared to conventional substrates. Effectively, a propionic acid yield of 84.4 C-mole % and a low acetic acid production (P/A molar ratio reaching 37) have been obtained from glycerol with P. acidipropionici (Barbirato, F., Chedaille, D. and Bories, A., “Propionic acid fermentation from glycerol: comparison with conventional substrates”, Appl Microbiol Biotechnol, 47, p. 441-446, 1997). This strain also produces some propanol from glycerol, indicating that when the substrate has a higher reduction level also products with a higher reduction level can be produced because of the better NADH/NAD+ balance.

Glycerol→propionate+1H₂O

Himmi et. al. compared the fermentation of glycerol and glucose and product formation for P. acidipropionici and P. freudenreichii ssp. shermanii. Fermentation end-products were propionic acid as the major product, acetic acid as the main byproduct and two minor metabolites, n-propanol and succinic acid. The yield of propionic acid was up to 79 C-mole % (64 wt %) with glycerol as the carbon source (Himmi, E. H., Bories, A., Boussaid, A. and Hassani, L., “Propionic acid fermentation of glycerol and glucose by Propionibacterium acidipropionici and Propionibacterium freudenreichiissp. Shermanii”, Appl Microbiol Biotechnol, 53, p. 435-440, 2000). Rumen microorganisms that ferment lactate via the dicarboxylic acid pathway, produce more propionate relative to acetate when hydrogen is added (M. Schulmanda and D. Valentino, “Factors Influencing Rumen Fermentation: Effect of Hydrogen on formation of Propionate”, Journal of Dairy Science, vol. 59 (8), p. 1444-1451, 1976). Acetic acid was almost eliminated when a high H2 pressure was applied during the fermentation with Propionispira arboris containing hydrogenase (Thompson T. E, Conrad R, Zeikus J. G., “Regulation of carbon and electron flow in Propionispira arboris: Physiological function of hydrogenase and its role in homopropionate formation”, FEMS Microbiol Lett 22, p. 265-271, 1984 and U.S. Pat. No. 4,732,855).

According to the Wood-Werkman cycle, endogenous CO₂ is released with acetic acid formation by Propionibacteria from glucose, lactose, or lactate fermentation (Deborde C., Boyaval P. 2000, Interactions between pyruvate and lactate metabolism in Propionibacterium freudenreichii subsp. shermanii: In vivo 13C nuclear magnetic resonance studies, Appl Environ Microbiol 66: 2012-2020). CO₂ can be fixed in Propionibacteria to form oxaloactate from PEP catalyzed by PEP carboxylase and then lead to succinate generation. Based on the metabolic pathway (Wood-Werkman cycle), CO₂ (HCO₃−) is required to convert phosphoenolypyruvate (PEP) into oxaloacetate by the enzyme phosphoenolypyruvate carboxylase. Through several sequential reactions, oxaloacetate is finally converted to propionic acid. In case of glycerol as substrate, nearly no acetate and hence CO₂ is produced. Applying an exogenous CO₂ pressure during fermentation has an positive effect on metabolite production rate and in particular a higher succinate accumulation thanks to the higher PEP carboxylation activity (“Effect of carbon dioxide on propionic acid productivity from glycerol by Propionibacterium acidipropionici”, An Zhang and Shang-Tian Yang, SIM annual meeting and Exhibition, San Diego, 2008).

Most propionic acid producing bacteria have the enzymes of the tricarboxylic acid cycle (TCA) which explain the variable P/A ratios for different strains. Some of the acetyl-CoA can be utilized in the TCA cycle by condensation with pyruvate into citrate (see FIG. 2). The end result is that more CO₂ is produced in the TCA cycle through the decarboxylations and less acetate is secreted. P/A ratios from 2.1 to 14.7 and CO₂/acetate ratio from 1.0 to 6.3 have been reported from glucose (Wood H G., “Metabolic cycles in the fermentation of propionic acid”, in Current Topics in Cellular regulation, Estabrook and Srera R W, eds., New York: Academic Press. vol 18, p 225-287, 1981).

Pelobacter propionicus, using the dicarboxylic acid pathway, has been show to grow on ethanol as substrate while producing propionate in presence of CO₂ (Schink, B., Kremer, D. and Hansen, T., “Pathway of propionate formation from ethanol in Pelobacter propionicus”, Arch. Microbiol. 147, 321-327, 1987 and S. Seeliger, P. Janssen, B. Schink, “Energetics and kinetics of lactate fermentation to acetate and propionate via methylmalonyl-CoA or acrylyl-CoA”, FEMS Microbiology Letters, 211, pp. 65-70, 2002). When ethanol is fed together with CO2 and hydrogen, significant amounts of propanol are produced. Ethanol is converted into acetyl-CoA (via acetaldehyde) while producing electrons for the carboxylation of acetyl-CoA into pyruvate, catalysed by pyruvate synthase. Combined with the dicarboxylic acid pathway propionate is produced from ethanol and CO₂ (Schink et al., 1987).

3 ethanol+2HCO₃−→2 propionate−+acetate−+H++3H₂O

Pelobacter propionicus is not able to reductively convert acetate and CO₂ into propionate whereas Desulfobulbus propionicus does make propionate from acetate and CO₂ (Schink et al., 1987).

acetate−+HCO₃−+3H₂ propionate−+3H₂O

Acrylate Pathway:

Though many bacteria can ferment a variety of substrates anaerobically into lactate as end product, some can further reduce the lactate into propionate, like Clostrium propionicum, Clostrium neopropionicum, Megasphaera elsdenii and Prevotella ruminicola (P. Boyaval, C. Cone, “Production of propionic acid”, Lait, 75, 453-461, 1995) by using the acryloyl-CoA pathway (see FIG. 3). Several substrates (sugars, ethanol and some aminoacids) that can be converted into pyruvate as intermediate can be further reduced into propionate as main product with acetate and butyrate as co-product. The key reaction is the lactoyl-CoA dehydration into acryloyl-CoA that is subsequently reduced to propionyl-CoA. The electrons for this reduction are provided by the oxidation of pyruvate/lactate into acetate and CO₂ (G. Gottschalk, “Bacterial Metabolism”, 2nd ed., Springer, New York, 1986).

Clostridium neopropionicum (strain X4), using the acrylate pathway, is able to convert ethanol and CO₂ into acetate, propionate and some propanol (J. Tholozan, J. Touzel, E. Samain, J. Grivet, G. Prensier and G. Albagnac, “Clostridium neopropionicum sp. Nov., a strict anaerobic bacterium fermenting ethanol to propionate through acrylate pathway”, Arch. Microbiol., 157, p. 249-257, 1992). As for the dicarboxylic acid pathway, the intermediate acetyl-CoA produced from the substrate ethanol is linked to the acrylate pathway via the pyruvate synthase that converts acetyl-CoA into pyruvate by carboxylation with CO₂.

Recently, an alternative route leading to acryloyl-CoA consists in the conversion of acetyl-CoA into malonyl-CoA by carboxylation with CO₂. The malonyl-CoA is further converted into acryloyl-CoA via four steps implicating malonate-semialdehyde, hydroxypropanoate, hydroxypropanoyl-CoA and finally acryloyl-CoA. Acryloyl-CoA produced by this pathway is subsequently reduced to propionyl-CoA similarly to the reactions leading to acryloyl-CoA by dehydratation of lactoyl-CoA (J. Zarzycki, “Identifying the missins steps of the autotrophic 3-hydroxypropionate CO₂ fixation cycle in Chloroflexus aurantiacus, PNAS, 106(50), p. 21317, 2009; I. Berg, “A 3-hydroxypropionate/4-hydroxybutyrate autotrophic carbon dioxide assimilation pathway in archaea, Science, 318, p. 1782, 2007).

Preferably, the symbiotic C3-producing microorganisms of the invention are capable of growing on ethanol and/or acetate as their primary carbon source. These microorganisms include, but are not limited to, Pelobacter propionicus, Clostridium neopropionicum, Clostridium propionicum, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii. In particular embodiments of the invention, the C3-producing microorganisms are propionogens. Propionogens refers to any microorganism capable of converting syngas intermediates, such as ethanol and acetate, to propionic acid and propanol. Propionogens of the invention utilize one of at least two distinct pathways for the conversion of syngas to propionate—the methylmalonyl-succinate pathway (shown in FIG. 2) and the lactate-acrylate pathway (shown in FIG. 3).

Through the symbiotic arrangement described herein, the anaerobic microorganism cultures of the present invention have the capability in a spatially separated symbiotic relationship to produce propanol from gaseous carbon and electron sources. Suitable sources of carbon and electron sources for the cultures include “waste” gases such as syngas, oil refinery waste gases, steel manufacturing waste gases, gases produced by steam, autothermal or combined reforming of natural gas or naphtha, biogas and products of biomass, coal or refinery residu's gasification or mixtures of the latter. Sources also include gases (containing some H₂) which are produced by yeast, clostridial fermentations, and gasified cellulosic materials. Such gaseous substrates may be produced as byproducts of other processes or may be produced specifically for use in the methods of the present invention. Those of skill in the art will recognize that any source of substrate gas may be used in the practice of the present invention, so long as it is possible to provide the C1-fixing microorganism cultures with sufficient quantities of the substrate gases, under conditions suitable for the bacterium, to carry out the fermentation reactions.

In one preferred embodiment of the invention, the source of CO, CO₂ and H₂ is syngas. Syngas for use as a substrate may be obtained, for example, as a gaseous product of coal or refinery residu's gasification. Syngas may also be produced by reforming natural gas or naphtha, for example by the reforming of natural gas in a steam methane reformer. Alternatively, syngas can be produced by gasification of readily available low-cost agricultural raw materials expressly for the purpose of bacterial fermentation, thereby providing a route for indirect fermentation of biomass to alcohol. There are numerous examples of raw materials which can be converted to syngas, as most types of vegetation could be used for this purpose. Suitable raw materials include, but are not limited to, perennial grasses such as switchgrass, crop residues such as corn stover, processing wastes such as sawdust byproducts from sugar cane harvesting (bagasse) or palm oil production, etc. Those of skill in the art are familiar with the generation of syngas from such starting materials. In general, syngas is generated in a gasifier from dried biomass primarily by pyrolysis, partial oxidation, and steam reforming, the primary products being CO, H₂ and CO₂. The terms “gasification” and “pyrolysis” refer to similar processes; both processes limit the amount of oxygen to which the biomass is exposed. The term “gasification” is sometimes used to include both gasification and pyrolysis.

Combinations of sources for substrate gases fed into the indirect fermentation process may also be utilized to alter the concentration of components in the feed stream to the bioreactor. For example, the primary source of CO, CO₂ and H₂ may be syngas, which typically exhibits a concentration ratio of 37% CO, 35% H₂, and 18% CO₂, but the syngas may be supplemented with gas from other sources to enrich the level of CO (i.e., steel mill waste gas is enriched in CO) or H₂.

In some circumstances the method benefits from exposing the C3-producing microorganism to carbon dioxide and hydrogen. It is also possible to produce the carbon dioxide and hydrogen by the exposure of the C1-fixing microorganism to the gaseous substrate.

The microorganisms of the present invention must be cultured under anaerobic conditions. As used herein, “anaerobic conditions” means the level of oxygen (O₂) is below 0.5 parts per million in the gas phase of the environment to which the microorganisms are exposed. One of skill in the art will be familiar with the standard anaerobic techniques for culturing these microorganisms (Balch and Wolfe, 1976, Appl. Environ. Microbiol. 32:781-791; Balch et al., 1979, Microbiol. Rev. 43:260-296).

Currently, no natural symbiotic pairings able to produce propanol or acid propionic from syngas have been found in natural environments. However, microorganisms from natural environments, when paired together under the correct nutrient conditions and selection pressures can be forced to form these “unnatural” metabolic symbiotic pairings which will produce propanol from syngas.

Symbiotic cultures for use in the invention can be generated in several ways. One approach involves using nutrient selection pressures to form a metabolic symbiosis between at least two of the microorganisms found in an environmental sample containing a mixed anaerobic microbial community. In this method, the only carbon and electron sources available for microbial growth are either syngas and/or syngas fermentation products, such as ethanol and acetate. Under these nutrient selection pressures, microorganisms capable of growing on these nutrients will be enriched. A variation of the process for forming symbiotic associations described above involves dilution. This process allows the very slow growing C3-producing propionogens in the sample to reach a higher cell density. Dilution of enrichment cultures can proceed with either a continuously fed anaerobic fermenter or manually through serial dilutions of enrichment samples. Both dilution techniques apply the same nutrient selection pressure of carbon and electron sources described previously. Another method for establishing a symbiotic association capable of converting syngas to propanol involves the growing of two or more defined cultures and establishing the pairing of these separate cultures. A person skilled in the art would appreciate that there are numerous methods of pairing two or more defined cultures. For example, one method involves first growing a known C1-fixing homoacetogen in a fermenter with syngas as the only carbon and electron source. This is referred to as the C1-fixing fermentation zone. In a preferred embodiment, the C1-fixing homoacetogen will produce ethanol from the syngas. At the same time, a known C3-producing propionogen culture is grown in a separate fermentor zone. This is referred to as the C3-producing fermentation zone.

Once the homoacetogen culture in the C1-fixing fermentation zone has reached steady state with respect to ethanol and/or acetate productivity, the ethanol and/or acetate from the C1-fixing fermentation zone is passed to the propionogen culture in the C3-producing fermentation zone. A suitable volume ratio of the C3-producting propionogen culture to the C1-fixing homoacetogenic culture is about 1:50-1:1, most preferable about 1:10-1:2. Preferably, the homoacetogen will produce two times more ethanol than acetic acid and have reached an optical density (OD) of about 1.0-10.0, most preferably 2.0-3.0, before media from the C1-fixing fermentation zone is transferred to the C3-producing fermentation zone. Additionally, it is preferable that the C3-producing propionogen will have an OD of between about 0.1-5.0, most preferable between about 0.4-1.5 at the time of media transfer. To promote the production of the propionate, the C3-producing microorganism will also receive a continuous supply of CO₂ and (H₂ or N₂). CO₂ and (H₂ or N₂) may be passed to the liquid media of the fermentation zone of the C3-producing microorganism. Alternatively, gaseous contact of the CO₂ and (H₂ or N₂) with the C3-producing microorganism may be established by the use of suitable means, such as a membrane supported arrangement as described herein. The culture medium from the C3-producing fermentation zone, containing the propionic acid produced by the C3-producing microorganisms, is then returned back to the C1-fixing fermentation zone. The C1-fixing microorganism will reduce propionic acid to produce propanol as a product.

Preferably, the cyclic transfer of culture media between the C1-fixing and C3-producing fermentation zones will create a continuous symbiotic environment in both fermentation zones. However, in the process of transferring culture media from one fermentation zone to another fermentation zone, a portion of the culture may also be transferred. For example in transferring culture media from the C3-producing fermentation zone, a portion of the propionogens may also be transferred to the C1-fixing fermentation zone. Alternatively, a cell re-cycle system can be used prior to transfer of fermentation broth from either zone 1 or zone 2 whereby the cells from respective fermenters are removed or concentrated from their broth before transferring to subsequent vessels. The collected cells are returned back to their respective fermentation vessels. Examples of cell recovery systems include in-line continuous centrifuges or tangential flow filtration units.

A suitable medium composition used to grow and maintain symbiotic cultures described above, includes a defined media formulation. The standard growth medium is made from stock solutions which result in the following final composition per Liter of medium. The amounts given are in grams unless stated otherwise. Minerals: NaCl, 2; NH₄Cl, 25; KCl, 2.5; KH₂PO₄, 2.5; MgSO₄.7H₂O, 0.5; CaCl₂.2H₂O, 0.1. Trace metals: MnSO₄.H₂O, 0.01; Fe(NH₄)₂(SO₄)₂.6H2O, 0.008; CoCl₂.6H2O, 0.002; ZnSO₄.7H2O, 0.01; NiCl₂.6H2O, 0.002; Na₂MoO₄.2H₂O, 0.0002, Na₂SeO₄, 0.001, Na₂WO₄, 0.002. Vitamins (amount, mg): Pyridoxine HCl, 0.10; thiamine HCl, 0.05, riboflavin, 0.05; calcium pantothenate, 0.05; thioctic acid, 0.05; p-aminobenzoic acid, 0.05; nicotinic acid, 0.05; vitamin B12, 0.05; mercaptoethanesulfonic acid, 0.05; biotin, 0.02; folic acid, 0.02. A reducing agent mixture is added to the medium at a final concentration (g/L) of cysteine (free base), 0.1; Na₂S.2H₂O, 0.1. The final pH target for this growth media can be adjusted between about 5-7, preferably between about 5.5-6.0. Medium compositions can also be provided by yeast extract or corn steep liquor or supplemented with such liquids.

During initial growth of the C1-fixing homoacetogen cultures, syngas is continuously sparged into the media to provide sufficient dissolved CO and H₂ concentrations to support cell growth and fermentation products, ethanol and acetic acid. Syngas addition to the C1-fixing-homoacetogen reactor continues throughout the process to maintain a healthy solventogenic syngas fermenting culture. Initial growth of the C3-propionogen requires the addition of ethanol and/or acetate to the media to support the growth of these microorganisms prior to the symbiotic pairing step. Concentrations between 1-50 g/L of ethanol, acetate or combinations thereof are fed to the media continuously, preferably, between 1-20 g/L and most preferably between 1-10 g/L. Once the C3-propionogen is combined with the C1-homoacetogen or fermentation broths, ethanol and acetate are no longer feed to the media. Because the fermentation zone method described above requires free interchange of the medium between the fermenters containing the C1-fixing and C3-producing cultures, both of the fermentation zones will use essentially the same composition for the medium and will operate under similar conditions of temperature and syngas present. In another embodiment, the medium is optimized independently for both fermentation zone while maintaining a healthy environment for both fermentation zone. In still another embodiment, a different syngas composition and/or concentration can be fed to both fermentation zones.

The methods of the present invention can be performed in any of several types of fermentation apparatuses that are known to those of skill in the art, with or without additional modifications, or in other styles of fermentation equipment that are currently under development. Examples include but are not limited to bubble column reactors, two stage bioreactors, trickle bed reactors, membrane reactors, packed bed reactors containing immobilized cells, etc. These apparatuses will be used to develop and maintain the C1-fixing homoacetogen and C3-producing propionogen cultures used to establish the symbiotic metabolic association. The chief requirements of such an apparatus include:
a. Axenicity;
b. Anaerobic conditions;
c. Suitable conditions for maintenance of temperature, pressure, and pH;
d. Sufficient quantities of substrates are supplied to the culture;
e. Optimum mass transfer performance to supply the gases to the fermentation medium
f. The end products of the fermentation can be readily recovered from the bacterial broth.

Each fermentation reactor may be, for example, a traditional stirred tank reactor, a column fermenter with immobilized or suspended cells, a continuous flow type reactor, a high pressure reactor, a suspended cell reactor with cell recycle, and other examples previously listed. Furthermore, multiple reactors of each type may be arranged in a series and/or parallel reactor system which contains any of the above-mentioned reactors. For example, multiple reactors can be useful for growing cells under one set of conditions and generating n-propanol (or other products) with minimal growth under another set of conditions.

In one embodiment, the C3-producing propionogen culture is first grown in a fermenter with a biofilm support material that is either stationary or floating within the reactor. US Patent Publication 20090035848, which is herein incorporated in its entirety, shows the use of floating support material in a moving bed bioreactor. An example of such support material is the Mutag Biochips. This method allows the C3-producing microorganism to first establish a biofilm on the carrier material thereby increasing the cell retention time versus the hydraulic retention of the fermenter.

In general, fermentation of the symbiotic culture will be allowed to proceed until a desired level of propanol is produced in the culture media. Preferably, the level of propanol produced is in the range of 2 grams/liters to 75 grams/liters and most preferably in the range of 4 grams/liters to 50 grams/liters. Alternatively, production may be halted when a certain rate of production is achieved, e.g. when the rate of production of a desired product has declined due to, for example, build-up of bacterial waste products, reduction in substrate availability, feedback inhibition by products, reduction in the number of viable bacteria, or for any of several other reasons known to those of skill in the art. In addition, continuous culture techniques exist which allow the continual replenishment of fresh culture medium with concurrent removal of used medium, including any liquid products therein (i.e. the chemostat mode). Also techniques of cell recycle may be employed to control the cell density and hence the volumetric productivity of the fermenter.

The transfer of the ethanol and/or acetate from the C1-fixing fermentation zone to the C3-producing fermentation zone may be accomplished in any manner that maintains the segregation of the different cultures. Media containing the ethanol or acetate may be filtered for removal of the C1-fixing microorganism and then transferred to the fermenter containing the C3-producing culture.

The C3 propionogen culture may benefit from the use of a stationary substrate such as a membrane upon which to retain the culture. Systems and processes for supporting microorganism cultures on membranes are shown in US Patent Publication 20080305540, which is herein incorporated in its entirety, where the microorganism reside in a fermentation liquid and form a biofilm for retention on a membrane substrate. In such arrangements the substrate or membrane may provide a convenient means for segregating the different cultures.

A preferred method of transferring the ethanol and acetate is through the use of a membrane type fermentation zone to retain the C3-producing microorganism in the pores of the membrane. US Patent Publication 20090215163, which is herein incorporated in its entirety, shows such a system and arrangement where pores of a membrane retain the microorganisms in a gas phase environment while liquid containing nutrients and/or substrates permeate to the microorganisms from the opposite side of the membrane.

FIG. 4 shows an embodiment of the invention utilizing a C1-fixing fermentation zone and C3-producing fermentation zone arrangement. In this embodiment, fermentation reactor 10, a planktonic fermentation reactor, suspends the C1-fixing microorganism in a liquid culture media and a membrane fermentation reactor 12 retains the C3-producing microorganisms. A feed gas comprising at least CO and H₂ enters fermentation reactor 10 though feed gas line 14. A gas injector 16 mixes the feed gas with a recirculating stream of culture media withdrawn from fermentation reactor 10 via a line 20 and circulated by a pump 18 to gas injector 16 via a line 22 and a line 24. Off-gas comprising primarily CO₂, H₂ and unreacted feed gas components exits the reactor via a line 26. A line 28 directs a portion of the liquid culture media to the membrane fermentation reactor 12 and into the lumen 30 of a hollow fiber membrane 32. Membrane 32 controls the permeation of the culture media from the lumen 30 across the membrane to its outer surface where a plurality of pores (not shown) retain the C3-producing microorganism in a gaseous atmosphere that fills annular space 34 and surrounds the outside of membrane 32. The gaseous atmosphere keeps the C3-producing microorganism exposed to a high partial pressure of CO₂ and H₂ while the permeation of the culture media provides ethanol and/or acetate along with other nutrients to the microorganism for the production of propionate. A gas input line 36 supplies CO₂ and H₂ containing gas to the annular space 38. The relative pressure across the membrane may be controlled to prevent the accumulation of excess liquid on the outside of the membrane in a manner described in US Patent Publication 20090215163.

The culture media of the C3-producing fermentation zone containing propionate leaves membrane reactor 12 via a line 40. If desired, all or a portion of the culture media may be withdrawn via line 42 for recover of proprionate from the culture media. In most cases, a line 44 will return the propionate containing media to the C1-fixing fermentation zone in fermentation reactor 10 for conversion of the proprionate to propanol.

Use of the membrane 32 eliminates the need for thorough separation of the C1-fixing microorganism from the culture media that circulates to C3-producing microorganisms. The membrane also serves as a barrier to sequester any C1-fixing microorganism that remains the culture media from contacting the C3-producing microorganism.

A line 46 withdraws a portion of the media culture from fermentation reactor 10 for the recovery of the products such as propanol and, optionally, ethanol and/or acetate. The products that are produced by the microorganisms of this invention can be removed from the culture and purified by any of several methods that are known to those of skill in the art. For example, n-propanol can be removed by distillation at atmospheric pressure or under vacuum by adsorption or by other membrane based separations processes such as pervaporation, vapor permeation and the like and further processed such as by chemical/catalytic dehydration to produce propylene. Recycled liquid from the separation of the n-propanol may contain significant quantities of ethanol and/or acetate which may be returned directly to the membrane reactor as part of the circulating culture media.

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined to provide additional guidance to the practitioner regarding the description of the invention.

### EXAMPLES

The Examples which follow are illustrative of specific embodiments of the invention, and various uses thereof. They are set forth for explanatory purposes only, and are not to be taken as limiting the invention.

### Example 1

### C¹³-Labeled Propionic Acid Conversion to Propanol

To demonstrate that homoacetogen cultures growing on syngas convert propionic acid to propanol and other fermentation byproducts, C¹³-propionic acid experiments were performed. C¹³-propionic acid was fed to homoacetogen culture, Clostridium coskatti, at a concentration of 100 mM in a serum bottle and incubated at 37° C. Samples were withdrawn from the serum bottles at 2 hrs, 24 hrs and 1 week. GC-MS was used to identify the products containing the heavy stable isotope C¹³. C¹³ products were found in the propanol peak and there was no propanol produced without the C¹³ label. In addition there were no other products formed that contained the C¹³ heavy carbon isotope or its mass fragments demonstrating that homoacetogens can reduce propionic acid to propanol and no other end products when growing on syngas.

### Example 2

### Propionic Acid to Propanol in Homoacetogen Fermenters

An ethanol producing homoacetogen fermenter was continuously fed propionic acid to investigate the rate and yield of propanol. The initial concentration of ethanol in the fermenter was 500 mmol/L before propionic acid feed was started. Concentrations of propanol reached 167 mmol/L in the fermenter at a feed rate of 200 mmol/L propionic acid. Residual propionic acid in the fermenter was 27 mmol/L; therefore the conversion efficiency to propanol was 97%. The concentration of ethanol in the fermenter steadily decreased as the concentration of propanol increased. At 167 mmol/L propanol the fermenter contained 250 mmol/L of ethanol. This ratio of alcohols demonstrates an electron balance based on the gas consumption rates of syngas in the fermenter. A production rate of propanol at steady state of 0.22 g/L/hr was achieved in the fermenter. The results show both high conversion efficiency and rates of propionic acid to propanol by homoacetogenic microorganisms growing on syngas. In addition, these results also showed no impact on syngas consumption with propanol concentrations as high as 10 g/L (167 mmol/L). These results demonstrate that in a co-fermentation with the homoacetogen partner such as C. coskatii propionic acid is readily converted to propanol and the residual acetic acid is recycled and converted to propanol by this symbiotic coculture.

### Example 3

### Propionic Acid Production from Ethanol by Propionogens in Fermenters

A fermenter was started with Clostridium neopropionicum growing on ethanol as the source of electrons and bicarbonate and ethanol as the source of carbon. Ethanol concentration in the media feed was 213 mmol/L. The fermenter reached a concentration of 89 mmol/L propionic acid, 5 mmol/L of propanol, and a residual ethanol of 27 mmol/L at steady state. This represented a conversion efficiency of 76% from ethanol to propionic acid based on a theoretical conversion stoichiometry of 1.5 moles of ethanol per mole of propionic acid produced. Other reaction products included acetic acid and small amounts of butyric acid.

These experiments demonstrate the feasibility of converting ethanol to propionic acid at high yields under syngas fermentation conditions.

### Example 4

### Propanol Production by Co-Culture of a Homoacetogen and a Propionogen that Uses the Acrylate Pathway

A homoacetogenic bacterial culture of C. coskatii, grown on syngas in a fermenter and producing ethanol and acetate was mixed in with an anaerobic batch (bottle) culture of C. neopropionicum, which has the lactate acrylate pathway, grown on ethanol and producing propionate and low levels of propanol. The co-cultures, in bottles, were incubated under syngas with pH adjustment by addition of a dilute sodium bicarbonate (NaHCO₃) solution. The initial ethanol concentration in the co-cultures was approximately 180 mM (8.3 g/L), which was derived from the syngas fermentation. The initial propionate concentration was ˜3 mM (0.22 g/L), which was introduced into the co-culture mixture with the C. neopropionicum culture medium. The co-cultures were grown under syngas atmosphere of initial composition of ˜38% CO, ˜38% H₂, ˜15% CO₂ and ˜9% CH₄. The pH was adjusted periodically to maintain the level at or above pH 6.0. After 48 hrs samples were taken and analyzed. The analysis showed that ethanol was consumed and propanol production peaked at 36 mM (2.2 g/L), a level 12 times the initial molar propionate concentration, demonstrating that the propanol was derived from the syngas-produced ethanol and was not just the product of conversion of the initial propionate present. The propionate concentration also increased under these conditions to 33 mM (2.4 g/L) at day-three of incubation (when the experiment was terminated). These results indicate that a co-culture of a solventogenic syngas-metabolizing homoacetogen and an ethanol-metabolizing propionate-producing anaerobic bacterium can produce propanol from syngas-derived ethanol at a significant yield.

### Example 5

### Propanol Production by Co-Culture of a Homoacetogen and a Propionogen that Uses the Acrylate Pathway

A homoacetogenic bacterial culture of C. coskatii, grown on syngas in a fermenter and producing ethanol was mixed with an anaerobic, batch (bottle), culture of Pelobacter propionicus, which uses the methylmalonyl—succinate pathway, grown on ethanol and producing propionate and low levels of propanol. The initial ethanol concentration in the co-culture was approximately 120 mM (5.6 g/L), the majority of which was derived from the syngas fermentation. The initial propionate concentration was ˜1.8 mM, which was introduced into the co-culture mixture with the P. propionicus culture medium. The co-culture was incubated in a bottle at 30° C. with agitation under a syngas atmosphere with an initial composition of approximately 38% CO, 38% H₂, 15% CO₂ and 9% CH₄. The initial pH of the co-culture mixture was adjusted to ˜7.0 by addition of a dilute sodium bicarbonate (NaHCO₃) solution. Samples taken for analysis at the end of an 8 day incubation period showed ethanol utilization and propanol production. Approximately 40% of the original ethanol present in the mixture was consumed (47.44 mM) which resulted in a final total C3 compound (propanol+propionate) concentration of 17.5 mM. Propanol represented the majority of the C3 production with a final concentration of 14.43 mM while the propionate concentration was 3.07 mM. These concentrations represent a 13 and 1.67 times increase above initial values for propanol and propionate, respectively and a net production of 14.56 mM C3 compounds. There was no net production of C3 compounds in a control experiment where the Pelobacter propionicus cells were not present. These results demonstrate that a co-culture of a solventogenic syngas-metabolizing homoacetogen and an ethanol-metabolizing propionate-producing anaerobic bacterium can produce propanol from syngas-derived ethanol at a significant yield.

### Example 6

### Propanol Production from Syngas Using C1 and C3 Fixing Organisms in Separate Bioreactors

In this experiment a known propionogen, Clostridium neopropionicum, was first seeded into the pores of a MSBR which was a hydrophilic polysulfone hollow fibers (Spectrum Lab, Model# M7-500S-300-01N) with spongy pore structure. The fiber has ID of 0.5 mm and OD of 0.66 mm. The membrane has nominal molecular weight cutoff of 500 k. The whole membrane module was 3.12 cm in diameter and 20.6 cm in length, with 0.41 m2 of total membrane surface area.

The propionogen was grown on a media consisting of minerals, vitamins and additional 0.5 g/L yeast extract and a continuous feed of approximately 0.2 g/hr of Ethanol. Typical composition of the media was:
Minerals: NaCl, 2; NH₄Cl, 25; KCl, 2.5; KH₂PO₄, 2.5; MgSO₄.7H₂O, 0.5; CaCl₂.2H₂O, 0.1.Trace metals: MnSO₄.H₂O, 0.01; Fe(NH₄)₂(SO₄)₂.6H₂O, 0.008; CoCl₂.6H₂O, 0.002; ZnSO₄.7H₂O, 0.01; NiCl₂.6H₂O, 0.002; Na₂MoO₄-2H₂O, 0.0002, Na₂SeO₄, 0.001, Na₂W04, 0.002.
Vitamins (amount, mg): Pyridoxine HCl, 0.10; thiamine HCl, 0.05, riboflavin, 0.05; calcium pantothenate, 0.05; thioctic acid, 0.05; p-aminobenzoic acid, 0.05; nicotinic acid, 0.05; vitamin B12, 0.05; mercaptoethanesulfonic acid, 0.05; biotin, 0.02; folic acid, 0.02.

A reducing agent mixture was added to the medium at a final concentration (g/L) of cysteine (free base). The pH of this experiment was started at 6. In addition to the defined media reagents, 0.5 g/L yeast extract was also added to supplement unknown nutritional components for the C3-producing bacteria.

After over 3 weeks of initial growth of the propionogen a homoacetogen, Clostridium autoethanogenum, was seeded into a fermentor that used a CSTR vessel. The media used for both the growth of the propionogen in the MSBR and the homoacetogen in the CSTR was the same as above.

Gas composition used on the shell-side of the MSBR was 20 mol % CO₂ and 80% N₂ flow rate 100 mL/min throughout the experiment. Gas composition for the growth of the homoacetogen in the CSTR was initially 7% CO, 34.5% H₂, 23.8% CO₂, 4.8% CH₄ and balance N₂, but then later switched to 56 mol % H₂, 21 mol % CO, 4.8% CO₂ and balance of CH₄.

The gas uptake at the homoacetogen fermentor increased and reached approximately 15 mmole/l/hr for both H2 and CO at approximately 900 hrs. At this time the product concentration (g/l) of the primary products were: ethanol=3.29; propanol=0.24; acetic acid=14.15 and propionic acid=0.52.

This established that the two stage bioreactor with the propionogen in one bioreactor and the homoacetogen in a separate bioreactor could be operated with syngas conversion by the homoacetogen to its primary products ethanol and acetic acid and the propionogen converting these to the C3 products propanol and propionic acid in a separate bioreactor.

1. A method for producing propanol comprising:
a) exposing gaseous substrates selected from the group consisting of carbon monoxide, carbon dioxide and hydrogen or combinations thereof to a C1-fixing microorganism, in a first fermentation zone, under conditions effective to convert the gaseous substrate into ethanol or acetate; and,
b) passing ethanol and/or acetate from step a) to a C3-producing microorganism, in a second fermentation zone, under conditions effective to convert the ethanol and/or acetate to propionate and/or propanol.

2. The method of claim 1 wherein the C1-fixing microorganism is a solventogenic acetogen, using the acetyl-CoA pathway.

3. The method of claim 1 wherein the C1-fixing microorganism is selected from the group consisting of Clostridium coskatii, Clostridium ljungdahlii, Clostridium authoethanogenium, Clostridium ragsdalei, Alkalibaculum bacchi, Clostridium thermoaceticum, and Clostridium aceticum.

4. The method of claim 1 wherein the C3-producing microorganism is a propionogen.

5. The method of claim 4 wherein the propionogen uses the lactate-acrylate pathway for the production of propionate.

6. The method of claim 4 wherein the propionogen uses the methylmalonyl-succinate pathway for the production of propionate.

7. The method of claim 1 wherein the C3-producing microorganism is selected from the group consisting of Clostridium neopropionicum, Clostridium propionicum, Pelobacter propionicus, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii.

8. The method of claim 1 wherein propanol is produced by passing at least a portion of the propionate from the C3-producing microorganism in the second fermentation zone to the C1-fixing microorganism in the first fermentation zone under conditions effective to produce propanol.

9. The method of claim 1 wherein the gaseous substrate is syngas.

10. The method of claim 1 wherein the pH of the C1-fixing microorganism in the first fermentation zone and the C3-producing microorganism in the second fermentation zone are maintained between about 5.0 to 7.0.

11. The method of claim 1 wherein the C1-fixing microorganism is maintained under planktonic conditions and the C3-fixing microorganism is maintained as cells fixed on a support.

12. The method of claim 1 wherein the C3-fixing microorganism is maintained under planktonic conditions and the C1-fixing microorganism is maintained as cells fixed on a support.

13. The method of claim 11 and 12 wherein the support for the cells comprises a membrane defining pores that retain the cell therein.

14. The method of claim 1 wherein the conditions effective to produce the propionate from ethanol and/or acetate with the C3-producing microorganism include exposing the C3-producing microorganism to syngas, at least containing carbon dioxide and hydrogen.

15. The method of claim 14 wherein the carbon dioxide and hydrogen is produced by the exposure of the C1-fixing microorganism to the gaseous substrate.

16. An anaerobic symbiotic system for conversion of syngas to propanol and/or propionic acid, the system comprising syngas, culture media, a C1-fixing microorganism in a first fermentation zone, a C3-producing microorganism in a second fermentation zone, a CO₂ and H₂ source and a transfer conduit for exchanging culture media between the C1-fixing microorganism in the first fermentation zone and the C3-producing microorganism in the second fermentation zone.

17. The anaerobic symbiotic system of claim 16 wherein the C1-fixing microorganism is a solventogenic acetogen, using the acetyl-CoA pathway.

18. The anaerobic symbiotic system of claim 16 wherein the C1-fixing microorganism is selected from the group consisting of Clostridium Coskatii, Clostridium ljungdahlii, Clostridium authoethanogenium, and Clostridium ragsdalei and Alkalibaculum bacchi, Clostridium thermoaceticum, and Clostridium aceticum.

19. The anaerobic symbiotic system of claim 16 wherein the C3-producing microorganism is a propionogen.

20. The anaerobic symbiotic system of claim 16 wherein the C3-producing microorganism is selected from the group consisting of Clostridium neopropionicum, Pelobacter propinoicus and Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii.

21. The anaerobic symbiotic system of claim 19 wherein the propionogen uses the lactate-acrylate pathway for the production of propionate.

22. The anaerobic symbiotic system of claim 19 wherein the propionogen uses the methylmalonyl-succinate pathway for the production of propionate.

23. The anaerobic symbiotic system of claim 16 wherein the pH of the culture media is maintained between about 5.0 to about 7.0.
